(19) United States
(12) Patent Application Publication
(10) Pub. No.: US 2006/0029644 A1
(43) Pub. Date: Feb. 9, 2006

(54) In vitro biochemical reactions with free anhydrous D-ribose

(76) Inventor: Keith Earl Kenyon, Van Nuys, CA (US)

Correspondence Address: Keith E. Kenyon, M.D., 4501 Nagle Avenue, Sherman Oaks, CA, 91423, US

(21) Appl. No.: 10/913,181

(22) Filed: Aug. 6, 2004

(51) Int. Cl. A61K 9/68; C07H 5/06

(52) U.S. Cl. 424/440; 536/55.3

(57) ABSTRACT

This invention discloses the spontaneous endothermic chemical reaction at room temperature that anhydrous D-ribose undergoes with one or more molecules, forming new molecules involving the ribose radical and also those reactions that require elevated temperatures for a rapid reaction, including amino acids that form protein and polypeptides, as well as just heating anhydrous D-ribose then cooling it to form the solid amber “rock candy” result.

### RELATED APPLICATIONS

This patent application is related to patent application Ser. No. 09/504,805 and its continuation in part Ser. No. 10/238,064, U.S. Pat. No. 6,553,254 B1, patent application Ser. No. 10/330,566, provisional patent application No. 60/488,914, provisional patent application No. 60/505,102, provisional patent application No. 60/534,272, provisional patent application No. 60/553,725, provisional patent application No. 60/561,489, provisional patent application No. 60/570,033, and patent application No. 10/886,070.

### FIELD OF THE INVENTION

This invention involves the fields of biochemistry, nutriceuticals, food, and medicine.

### BACKGROUND OF THE INVENTION

Patent application Ser. No. 10/886,070 “New Molecules as Produced by Endothermic Chemical Reactions with Anhydrous D-Ribose” disclosed how anhydrous free D-ribose produced by the fermentation of corn syrup is unstable until it is made into other molecules including its monohydrate, a much more stable form. To become more stable at room temperature, its anhydrous form undergoes spontaneous endothermic reactions, the most basic one by mixing one mol of free D-ribose and one mol of water. Unlike the original powder, the reaction result is a very viscous, sweet amber substance that is in effect a paste, becoming soft, even somewhat cheese-like in texture. Although the radical of ribose as a precursor to make adenosine triphosphate (ATP) has been known for decades, the nature and properties of the more stable molecule in vitro have not been disclosed until patent application Ser. No. 10/886,070 and its provisional applications were filed.

With respect to mammals, consumable in vitro free molecular anhydrous D-ribose does not exist and is not in the ambient environment or in the mammalian body, although its radical is universally present, being respectively the “D” and “R” of DNA and RNA, as well as numerous other molecules ordinarily made in vivo by glucose having a carbon atom removed in the pentose phosphate pathway (PPP) in the mitochondria to form the ribose radical. Anhydrous D-ribose only recently has been available commercially in large quantities by a fermentation process that uses the energy provided by corn syrup to enable bacteria metabolically to produce this unstable free molecule in its prior-to-a-reaction in vitro form. The chemistry that makes this anhydrous molecule in its free state has been worked out independently of this disclosure. What was not disclosed is the propensity of this molecule to react with other molecules due to its predisposition for endothermic chemical reactions, preventing it from being available as a pure sugar in plants like sucrose is. Before patent application Ser. No. 10/886,070 was filed, it was not disclosed nor realized by those skilled in the art that anhydrous D-ribose is not the best form for market, as it cannot even be heated to 120° F. without losing its powdery form. If observed, it was never understood nor disclosed, although of profound importance to disclose. Disclosure would create opportunities to understand the anhydrous powder so as to take advantage of its in vitro endothermic chemistry and simply create the monohydrate in the first place. Furthermore, the endothermic nature of anhydrous D-ribose in vitro can form many new molecules besides its monohydrate, including for in vivo use.

The monohydrate is an entirely new molecule with chemical and physical properties that are entirely different from its anhydrous precursor, being more compact and stable. Therefore, economically the anhydrous powder as a powder is best used in a combination only for the convenience of making the composition prior to the ribose being rendered as its monohydrate as will be disclosed herein. The fact that the powder quickly becomes something else, different both in physical and chemical properties, needs to be realized. Anhydrous D-ribose should not be put into any composition that defines or labels the molecule as being stable. It cannot be defined molecularly as only being C₅H₁₀O₅ as glucose is defined, C₆H₁₂O₆, because it is unlike glucose and can change quickly. Glucose is exothermic in vivo in the mammalian body including man and is not endothermic in vitro. On the other hand, ribose is endothermic in vivo and in vitro, but its in vitro nature, subject to spontaneous endothermic chemical reactions, has been disclosed only herein and in prior disclosures by this inventor.

Knowing the endothermic chemical reaction capability of D-ribose, will open up its remarkable in vitro chemistry. Nevertheless, knowing its in vitro chemistry requires correct labeling. The powder must be labeled as unstable, especially if marketed ultimately for cooking. It cannot be combined with other nutrients, food, or even by itself without warning of its instability even with a small amount of heat. Yet ribose is a very important nutrient in spite of its marketing being hindered as a result of its instability not being recognized and taken advantage of by those skilled in the art. To clarify how a more stable form of ribose can be incorporated into many such compositions so as to make them possible is a purpose of this disclosure as well as to awaken interest in the incredible possibilities that lie undiscovered and not researched about the endothermic nature of in vitro D-ribose (in vitro herein means D-ribose outside the bodies of mammals in its free form). It is obvious that our bodies need a great deal of the ribose radical. We produce all we can use within our bodies. Our pentose phosphate pathway (PPP) uses glucose's exothermic nature in vivo and ribose's endothermic nature to enable both exothermic and endothermic reactions to take place which enable glucose to be phosphorylated and a carbon atom removed ultimately to become a phosphorylated form of ribose that then can help form adenosine triphosphate (ATP). The radical of ribose is also vital to make DNA, RNA, and various forms of niacinamide adenine dinucleotide (NAD) which vital molecule is half ribose. We often can't produce enough of the ribose radical, especially for energy, because it takes in the vicinity of at least three days to make it and every cell needs ATP. Some, especially the muscles, need very large amounts of it. Nevertheless, in vivo ribose made in the PPP and in vitro anhydrous ribose powder made from a fermentation process are very different, because in vivo ribose always has another molecule or radical attached to it, whereas the pure product prepared for distribution to consumers has no such molecule or radical attached. In view of its reactivity in vivo and in vitro, ribose is one of the most unstable and reactive substances ever classified as a nutrient by the FDA.

Sometimes this instability can form an unsatisfactory combination that is different from what is intended. In vitro ribose has lacked satisfactory chemical investigation with results disclosed. The product that is sold as ribose today is the anhydrous D-ribose powder. The moment it is removed from the container and used for any purpose including ingestion, it undergoes a chemical reaction. Therefore, it must be stored alone, kept separated from other nutrients, not heated, and be so labeled, or it may react vigorously and variously, according to the molecule it is kept with or the use for which it is intended. This extreme reactivity, so different from that of glucose and other such sugars, is the reason life as we know it exists, but because of this reactivity, ribose presents many problems in its packaging and marketing that have been poorly understood by those skilled in the art.

Bioenergy, Inc. began producing pure in vitro anhydrous D-ribose in commercial amounts by 1999 and felt ribose had great promise but failed to research its in vitro chemistry so as to enable there to be minimal problems. The research or lack thereof that went into its assigned U.S. Pat. No. 6,159,942 issued in December of 2000 becomes apparent by the promises it does not fulfill. It refers to ribose as having a “pleasant” taste but does not disclose its sweetness. The anhydrous powder as a powder is not very sweet until its chemistry is changed due to its hydration in the mouth. Its inventors did not know that anhydrous D-ribose is endothermic, with this lack of knowledge still printed in the PDR purchasable in 2004. In error the Bioenergy patent combined L-carnitine with D-ribose in an unstable mixture. It also made the incorrect assumption that creatine is stable when mixed with ribose by not disclosing that its basic commercial form, creatine monohydrate, is unstable with the preferred vehicle of the invention, water. It further failed to disclose that such powdered anhydrous D-ribose itself is unstable at 150° F. or even below, becoming an amber paste unlike glucose, sucrose, lactose, and fructose which remain powders when heated alone or mixed with each other at 150° F. Mixing anhydrous D-ribose with any of them and adding just a little heat causes a profound change, and they no longer are two powders, nor even one. In spite of the promise of U.S. Pat. No. 6,159,942, it is mostly inoperative, because the in vitro chemistry of anhydrous D-ribose, was not conducted nor disclosed if it were conducted. Molecules formed by this endothermic chemical reaction were not obvious.

Nevertheless, Bioenergy marketed D-ribose to those with heart trouble in a composition called Corvalen™, containing the amino acid, L-arginine 2 g, anhydrous D-ribose 5 g, dextrose 5 g, Vitamin C 90 mg, Vitamin B-6 3 mg, folate 200 mcg, Vitamin B-12 30 mcg, without realizing and investigating the endothermic reaction that was caused. Directions were to mix the product with eight ounces or approximately 240 ml of cold water. The mixture was unstable from every perspective, L-arginine being the most unstable, becoming a dark chocolate-colored substance when placed into an oven at 150° F. even for a short time, starting to turn dark even before that temperature is reached. Vitamin C and dextrose ordinarily remain in the powder form if reacted with each other at 150° F. but not if in vitro anhydrous D-ribose is present. The endothermic reaction was not obvious then and still is not obvious except to this inventor.

Bioenergy withdrew the Corvalen composition from the market and switched to ribose alone in 2002 and has only done clinical studies, mostly with anhydrous D-ribose for congestive heart failure, an admirable pursuit, but requiring this inventor to do the basic research on the chemistry of the reactive powder, discovering that free D-ribose will react with many chemicals besides becoming a monohydrate. This brings up a perplexing problem. Each new previously undisclosed molecule becomes an entirely new substance with much stronger chemical bonds and far different physical properties. Even though new molecules are formed, some of which may have health and pharmaceutical benefit, failure even to understand the monohydrate retards the chemistry of this strange highly reactive molecule that is created uncombined only by a fermentation process. Even here the amber colored monohydrate “contamination” is feared, not researched, understood, nor embraced as is the reason for this disclosure.

Clearly there is a chemical as well as physical difference between in vitro with respect to mammals, anhydrous D-ribose and its monohydrate, including the monohydrate's greater sweetness and stability. This can be exploited in certain nutrient combinations including with creatine monohydrate. Thus those skilled in the art were unable to take advantage of the chemical and physical properties of ribose monohydrate because of lack of timely disclosure. Bioenergy was probably unable to understand the chemistry, so did not disclose that the monohydrate existed, rather than fear to disclose it because its existence would “damage” the powder to which the company was beholden. For whatever reason this conduct prevented Bioenergy from giving D-ribose greater ability to be marketed and thereby to bring down its price so its endothermic properties would be available to the many rather than the few, which this disclosure seeks to remedy.

It would be logical to assume that the dextrorotary form of ribose is retained by its monohydrate. The newly discovered molecule made by the endothermic reaction of ribose with water at room temperature is a very viscous, sweet, paste-like substance, occupying considerably less volume than the powder. Difficulties that its structure imposes need to be overcome so that advantage can be taken of the benefits it offers, and new ways to combine it with foods and nutrients, as well as new drugs, can be made.

This invention is designed, among other things, to overcome the deficiencies of previous applications and inventions by disclosing the in vitro endothermic chemistry of free anhydrous D-ribose after it is formed by fermentation so it can be used to form the myriad of new molecules possible.

### BRIEF SUMMARY OF THE INVENTION

This disclosure seeks to make use of one or more products resulting from 1) a room-temperature spontaneous endothermic chemical reaction of the anhydrous solid, marketed as a powder from a fermentation process, D-ribose, in vitro with respect to mammals, and water on a mol per mol basis of each in direct multiples which then can be an alternative to anhydrous D-ribose and 2) under elevated temperatures, the aforementioned powder reacting with other molecules including water vapor. Let's discuss the first.

When increased sweetness is needed or for new applications that the physical and chemical nature of unstable anhydrous D-ribose precludes, its monohydrate may be employed. The first product of this disclosure, made at room temperature is a viscous sweet amber opaque substance that is highly soluble in water. In making opaque ribose monohydrate, care must be taken not to have an excess of ribose or an excess of water in the mix. Subjected to heat the translucent form will appear. It must be kept in mind that anhydrous D-ribose placed into the mouth in powdered form is converted to the monohydrate immediately, but without this disclosure and this inventor's preceding disclosures concerning this endothermic reaction, this powder was not disclosed by label and literature to be physically and chemically unchanged when ingested. Ironically, although pleasant to taste as a powder, only the part that is closest to the tongue is very sweet, because that part has been changed to the sweet monohydrate. Converting the anhydrous powder to the monohydrate is required when sweetness is important, especially for things like candy or to mask the taste of more bitter compositions.

Unlike ordinary food sugars, such as dextrose, fructose, sucrose, and lactose, anhydrous D-ribose is not a stable a sugar in vivo or in vitro. If used in cooking it will react chemically and perhaps unfavorably with some kinds of food. Using the sweet more stable ribose monohydrate presents no metabolic disadvantage at room temperature or body temperature, and it is what the anhydrous powder becomes when it is placed in the moisture of the mouth and stomach. Since ribose has only recently been manufactured for supplementary purposes, it was not realized until this disclosure and its pertinent predecessors that upon being ingested, it logically forms a quickly absorbable monohydrate instead of reacting directly with food molecules in the intestines or in the blood stream. It is fortunate that its chemistry and endothermic reactivity exists and is a tribute as to how resourceful this molecule is in making life possible. Key to the ATP reaction is for one ribose carbon atom to react with a nitrogen atom with respect to the purine, adenine, but also to remove another water molecule to make an ester with phosphoric acid attached to another carbon atom, yielding adenosine monophosphate (AMP) in the PPP and ultimately ATP. This in vivo reaction involving the formation of both a ribosyl and an ester (riboside) by the ringed molecule of D-ribose demonstrates its unique versatility.

D-ribose being very endothermic in nature, the ribose radical in the body is enabled to react with amino acids, purines, phosphates and many other molecules in endothermic in vivo reactions. It was not obvious to those skilled in the art that in vitro D-ribose's extreme instability should be investigated, and no such disclosures appeared in the PDR and the Internet. It was not until the disclosures of this inventor have been made that there has been any attempt to define these endothermic reactions. With respect to the amino acids that make up protein in the body, such as arginine and carnitine, a reaction between at least some of them and anhydrous D-ribose takes place rapidly at temperatures even as low as 150° F. These result in presumptive ribosyls or ribosides. Whether the likely resulting compounds of these endothermic reactions between two solid powders would be ribosides or ribosyls depend on whether the resulting bond is C—C, C—N, or C—O—R. The reaction between anhydrous D-ribose and NaPCA as disclosed in provisional patent application No. 60/570,033 and patent application Ser. No. 10/886,070, is also presented with the same two likely molecular structural possibilities. Since the ribose ring can react in both ways and in some cases, both in the same compound, the same kind of chemical reactions can be used to obtain many new molecules, first in the test tube and then, depending on their properties, for use in the general population when applicable.

Either way they should have great promise in pharmaceutical investigation. They are either entirely new molecules made as a result of an endothermic reaction in vitro or are a new way to produce such molecules, if known, at far less cost because of the endothermic reaction. Nevertheless, in order to provide the funds to research these new molecules and others, we must initially explore ways D-ribose monohydrate, can be used, including for more stable compositions than using its anhydrous precursor.

The hydrate of D-ribose starts to form whenever ribose is presented to water including water vapor and steam. D-ribose is really not a nutrient. Although it is produced by a natural fermentation process, it is not normal to the diet just as ethanol is not. Although the chemistry has not been obvious to those skilled in the art, it is still logical that D-ribose would not behave like a plant nutrient such as sucrose, and ribose doesn't. Ordinary free sugar remains the same molecule before being put into the mouth as immediately afterward, but not free ribose. Its natural most stable molecular state inside the body, prior to forming a radical for another molecule, is to form its monohydrate, so it would make sense to produce it in its more stable amber form, either opaque or more translucent depending on its projected use in combination with other consumable ingredients, even with ordinary sugar.

This new art, enabling ribose monohydrate to be used in such preparations requires special methods due to its varying nature. Ribose monohydrate can be made in two ways. The first way at room temperature is to add multiples of a mol of water to the same multiples of a mol of ribose and get the opaque amber paste. Package it the same way jellies, yogurt, sour cream, etc. are packaged. On the other hand, just subjecting in vitro anhydrous D-Ribose to heat causes it to form the same monohydrate molecule but this time more translucent and much harder than the opaque after cooling. Only a moderate increase in heat is needed to package the soft kind like sour cream so its cost-effectiveness is accommodated. The extreme solubility of ribose monohydrate will mean that any remnants in the empty container can be used by simply putting water into such container and drink the remnants. Also a small amount of water can convert both the opaque and more translucent forms into a viscous amber solution that can be poured out of a container rather than scooped out or used as a solid.

If it is desired for it to be less viscous but still sweet, mixing ribose monohydrate with an equal weight of water will make a very sweet drink from the start. Once again, any remnants can have water added and the resulting solution drunk for maximum cost effectiveness, especially for those who need it for their hearts and want the greatest value for what they pay. Dilutions beyond equal weights of the monohydrate and water would eliminate those properties of ribose monohydrate that are required for optimum sweetness and compactness, but the drink should be properly labeled as to actually what it contains chemically, the monohydrate of D-ribose, just like the monohydrate of creatine is so labeled. A level teaspoonful of D-ribose monohydrate compared to a level teaspoonful of anhydrous D-ribose would be more concentrated in the case of the pure monohydrate, so the monohydrate would weigh considerably more. If desired, the soft opaque form, preferably to the hard translucent, is mixed with another liquid or a powdered substance and this will be discussed.

We can also produce ribose monohydrate from a mix with the anhydrous powder and other ingredients. After thorough mixing of the anhydrous ribose powder and the other powder or powders, add a mol of water per mol of ribose in any desired sequence and again mix thoroughly after each sequence. More than a mol of water can be added when more fluidity is needed if extra water is not contraindicated. The monohydrate, thus formed, will not slowly change the color of the composition with its associated ingredients as when the anhydrous powder is used.

When anhydrous D-ribose is made into the opaque monohydrate by adding a mol of water at room temperature, the result is amber but not translucent. The more attractive translucent substance can be accomplished by applying heat to the powder, although less translucent after cooling. To convert solid ribose monohydrate into an aqueous solution, only a small amount of water is needed. It takes a period of time for the solid monohydrate to go into an aqueous solution, especially if a concentrated one is desired. If left to stand after heating, this monohydrate remains sweet with increased hardness but does lose clarity. When we are dealing with ribose monohydrate in a manufacturing process, cooking, or producing a nutrient formula, excess water in the mix can react with water-unstable nutrients like creatine monohydrate, but excess anhydrous ribose can also react unstably with creatine monohydrate gradually forming ribose monohydrate.

There are three ways to make D-ribose monohydrate in a process outside the body (in vitro) to make a product which can be used in the body (in vivo). The first is to add a mol of water to a mol of in vitro anhydrous D-ribose in direct multiples of each and get the sweet amber opaque paste as discussed above. This room temperature ribose monohydrate, the most natural room temperature way the product exists, is best marketed as a paste the same way as are sour cream, yoghurt, peanut butter, etc. On the other hand, by heating the anhydrous powder a much harder translucent substance with variable clarity forms due to water vapor being more active at higher temperatures than liquid water. A third way is to use forced steam.

The monohydrate is sticky at room temperature but not near 0° C. A grated product could be produced like grated cheese is produced. The grated product is quite sweet and molecularly stable and can be added to any other food product to enhance its sweetness or its energy-precursor factor to make ATP as long as it is added and mixed under refrigerated conditions, but then the mix can be stored at room temperature. Another way is to thoroughly mix anhydrous D-ribose with a water-soluble substance, and then add a mol of water per mol of ribose and thoroughly mix the combination. Sucrose, glucose, and fructose would be good candidates, as well as some sugar substitutes. Since anhydrous ribose is endothermic with respect to water, the water will combine with the ribose and not with an ingredient that is only water-soluble. Of course, at elevated temperatures, in vitro anhydrous D-ribose will react directly with water vapor. Arginine and others actually become new molecules with ribose. Intent to market them would need the Food and Drug Administration (FDA) approval following application for a new drug study. The same is true for all such ribosyls, ribosides, and any and all such new molecules proposed for eventual human consumption or contact with the skin and environment.

An additional way is to mix thoroughly anhydrous D-ribose with a water insoluble substance such as creatine monohydrate, soy protein, whey, colostrum, a combination of these, or others. Then with respect to the mass of D-ribose, add the mass of water in terms of their respective molecular weights and again thoroughly mix. A paste or powder can result. Water can be added to the mix, if not water reactive, to make a sweet product with precursor means to make ATP more directly than the entire PPP. Of course, the order in which they are mixed can be important as will be discussed next.

The features of the present invention which are believed to be novel are set forth with particularity. The present invention, both as to its organization and the manner of operation, together with the further objects and advantages thereof, may be best understood by reference to the following exemplary and non-limiting detailed description of the invention, wherein;

### DETAILED DESCRIPTION OF THE INVENTION NO DRAWINGS

The first preferred embodiment is to enable the endothermic chemical reaction to occur either spontaneously at room temperature when a mol of in vitro, with respect to mammals, anhydrous D-ribose and a mol of water in direct multiples of each are mixed together or as an alternative, heating the anhydrous powder. At room temperature the first reaction results in a soft amber opaque paste and the second transforms into a sweet translucent amber solid that slowly hardens into more opaque rock-like candy. Each different endothermic reaction of in vitro anhydrous D-ribose forming its monohydrate may result in new in vitro molecules. Both forms of monohydrate become viscous liquids at elevated temperatures and are very soluble in water. Otherwise they have different properties, requiring different packaging and mixing with other substances. By way of example to get the opaque substance at room temperature, one kilogram of anhydrous D-ribose is reacted with 120 milligrams of water. Such a reaction results in an amber opaque paste that we would assume retains its dextrorotatory form. Regardless of whether or not the resulting molecule is designated as a monohydrate, by definition of such a chemical reaction, the molecule is different from either D-ribose or water. This new molecule and its properties have not been studied in vitro until now, but its use must be predicated on its properties. As with all new molecules, it needs to be studied, defined, and ways to overcome any difficulties or prejudices by way of mixing, packaging, and even toxicity resolved. If heating is not desired to make the monohydrate, the opaque quality can be changed to a translucent fluid by dissolving it into a small amount of water. The opaque paste if converted into a translucent viscous fluid will tend to retain increased translucence if extra water remains. If anhydrous conditions are needed for a composition, increased fluidity for ease of handling can be had by heating the original anhydrous ribose powder of the preferred embodiment and mix in the resulting monohydrate at elevated temperatures. Both the opaque and translucent forms will slowly re-solidify at room temperature, but the heated form will remain more translucent. Both require about half the packaging density of the powder but each needs to be packaged differently.

Since raising or lowering the temperature, even freezing the resulting molecule does not restore it to its white anhydrous precursor, the molecule has been changed permanently and must be packaged appropriately. Since the powder is so unstable and the monohydrates so different in properties both physical and chemical, the powder for use in cooking, food preparation, or mixing with other nutrients, should clearly be labeled as unstable with anything it comes in contact with, because once outside the package it starts to become the monohydrate even when mixed with other sugars. The monohydrate is far more stable, especially with water, sugars, and some consumable monohydrates, not itself, but it should be labeled for what it is and tested with each substance it comes in contact with. Whereas, the anhydrous ribose molecule is inherently unstable, the monohydrate is not.

D-ribose has been classified as a nutrient by the FDA and is being so marketed by Bioenergy, Inc. as well as a medical food without disclosing its unstable nature. The fact is that ribose is the most fundamental multi-carbon molecule in the body and every cell makes it in its mitochondria. It is of extremely low toxicity when taken in 20 grams or less a day. Therefore, it can be added to food or certain nutrient formulas, but if not in its more stable monohydrate form, calling attention to its increased instability is mandatory.

If packaged and marketed as the more stable monohydrate, it must be prepared for each general use in a way that accommodates the differences encountered per use. To make it stable, its molecular hydration in the endothermic reaction occurs at room temperature in one way and at elevated temperatures in another. Either way it no longer is a powder and cannot so act, but it can act as a sweet solid or semisolid substance with the highest possible solubility in water. It can be dissolved in a small amount of water and marketed as a liquid, but to make it into a particulate substance in its solid form is difficult because it is sticky at room temperature. At higher temperatures ribose monohydrate, whether formed opaque or translucent, is a viscous fluid but forms an amber translucent substance when cooled to room temperature again. At 0° C. the monohydrate becomes quite hard and stickiness disappears. Unlike water at 0° C., ribose monohydrate is not crystalline when cooled to that temperature, so it cannot simply be fractured and crushed at such cost-effective temperature levels for manufacturing, so it would need to be grated. The problem is because of stickiness, grated particles need to be made from the “frozen” monohydrate. This would make it necessary to store such particles at refrigeration temperatures. Cost-effectiveness plays an important role here. Both the opaque and translucent substance is directly useable. Furthermore, by adding a small amount of water, less than a mol of water compared to a mol of ribose monohydrate, both are converted into a more fluid liquid as described herein. Therefore, there are a large number of mixing choices to be made and each depends on cost-effectiveness of the mix and palatability of the product.

For industrial purposes, if elevated temperatures are contraindicated, frozen particles can be mixed quickly with another powder or powders in a cold room. The grated monohydrate must be kept refrigerated until mixed with a suitable powder, but once the mix is made the frozen particles will blend in with the overall mix, and it can be stored at room temperature as is the case also when the elevated temperature protocol is able to be used with heat-stable situations. Thus ribose monohydrate can be utilized at low or elevated temperatures but for mixing purposes, higher temperatures only if heat does not destabilize a composition. At higher temperatures ribose monohydrate becomes a fluid that can readily mix with powders. Anhydrous D-ribose is changed to the monohydrate at room temperature for more control in making sure that the monohydrate does not attract extra moisture from the heated air. Mixing it with other substances that are stable with it at 150° F. can be done at the higher temperatures for all or at room temperature since the originally opaque monohydrate returns to the solid state very slowly. By this procedure all powders can be thoroughly mixed into the ribose monohydrate fluid and packaged at room temperature which can be compared for cost effectiveness with mixing entirely at room temperature.

When it is desired to perform mixing at room temperature, adding extra water so as to put ribose monohydrate into a liquid state is the easiest way to do so. An aqueous solution of ribose monohydrate has some limitations that need to be addressed. It cannot be added to substances that are unstable in water such as creatine monohydrate. Although it will slowly degrade into the non-toxic monohydrate, anhydrous D-ribose can be used with proper labeling and instructions to expect it to change physically if heated. Otherwise, D-ribose monohydrate needs to be mixed with such creatine directly. Ribose monohydrate can be directly mixed with creatine monohydrate since neither molecule has free water. The resulting mix if more or less equal in amount is sweet tasting but a non-uniform paste results. Since creatine monohydrate is a powder and ribose monohydrate a paste, “drying” powders, which are substances that are able to disperse paste-like ingredients within the combination need to be used. In practice non-reactive sugars like sucrose and dextrose make the best dispersing molecules for ribose monohydrate. They are simple carbohydrates also and perhaps the monohydrate molecule disperses inside their crystals more naturally than it does with molecular structures that are dissimilar even though ribose monohydrate appears not to be crystalline. Nevertheless, suitable non-sugar substances can be used as will be disclosed.

It may be more convenient to use anhydrous D-ribose as the starting substance when mixing with a water-unstable as well as the unstable anhydrous ribose such as with creatine monohydrate, because it is better to mix two powders at cost-effective room temperature than a powder with a paste. Some elements of how to do this have been disclosed in patent application Ser. No. 10/886,070 but will be further disclosed herein to make a powder rather than a paste. To use ribose monohydrate at room temperature with the end product a composition precluding free water being present, it is necessary to make the ribose monohydrate as part of the mixing process, and this involves using anhydrous D-ribose in its powder form and once the first mixture has been made, add a mol of water per mol of ribose and again mix the combination. Because of the stickiness resulting in pastiness of ribose monohydrate extending to a mixture with creatine monohydrate, if there is to be a powder formed, a “drying” agent needs to be added at some point. These are substances that can “dry out” the mix and leave a powdery result. Dextrose, fructose, or sucrose can be used with their presumed dietary disadvantages, but ribose monohydrate is also very sweet. If the presently accepted standard dosage for weight lifting at 10 grams of creatine monohydrate shortly after a workout is used, and since 5 grams of D-ribose is considered effective when “stacked” with 10 grams of creatine monohydrate, using a sugar such as sucrose for the “drying” agent can enable a sweeter powder to be made rather than using protein powder. With respect to creatine monohydrate and substances like it, D-ribose can be used with proper labeling without adding a mol of water per mol of ribose.

On the other hand, the addition of a protein powder such as whey or soy works well, soy mixing better than whey but whey tasting better although more expensive than soy. Therefore, to make this kind of ribose-creatine powder, we would mix anhydrous D-ribose with creatine monohydrate and the “drying” agent in this case preferably soy protein if protein is the drying agent because it appears to “dry” better, if better taste is not paramount. Equal parts of each may be mixed together, or some other sequential combination of parts. Thorough mixing is required with both a protein or a sugar. At some point in the overall procedure exactly one mol of water per mole of anhydrous D-ribose is added followed by more mixing. Again the entire ultimate contents are thoroughly mixed. Another way to do this is to mix the anhydrous D-ribose with the protein powder or ordinary sugar or both, then add the mols of water proportional to the mols of ribose and thoroughly mix again. After this, if so desired, add another molecule like creatine and again thoroughly mix. Although this is one extra step, it may make a faster overall mixing process. Either technique can be used as with the next group of mixtures based on their solubility with water.

While most of the above disclosed preparations will be in the category of nutriceuticals, D-ribose in its monohydrate form can better be used with food if properly labeled. Because it does not ordinarily provide calories or carbohydrates as it is an end product of energy metabolism, it saves the body the need for such calories to be spent converting glucose into ribose. The immediate sweetness of ribose monohydrate, not having to be converted to the monohydrate in the mouth, coupled with its stability would enable it to simply be a sweetener like sucrose and fructose, but its high cost prevents its mass production for such a use until its cost comes down. Then it can be so used because of its non-food-carbohydrate, non-calorie attributes. In the meantime it can be used in upscale foods, and health-oriented candy is the most suitable such food to be marketed. The sweet rock-type candy of ribose monohydrate formed by heat only and correctly wrapped to accommodate any viscous fluid reformation and flavored as and if desired, is simple to make.

The fact that ribose monohydrate is sweet is a benefit and so is its stability when placed in contact with stored nutrients and food to enable it to be used in many upscale prepared nutrients and food that anhydrous D-ribose would tend to react with chemically. It can be combined with whey or other kinds of milk protein or with soy protein. Let us use the example of soy protein. If 200 grams of anhydrous D-ribose are mixed with 48 grams of water, 24 of those grams of water will react with the 200 grams of D-ribose and the other 24 grams will put the ribose monohydrate into a concentrated aqueous amber solution. Then if 200 grams of soy protein are added and mixed, a uniform suspension of the protein in the ribose monohydrate solution will occur. If the water is driven out by heat or this suspension is allowed to dry out over time, in either case, a mixture of ribose monohydrate and soy protein can be rendered a powder. Whey protein can be substituted for the soy with the same results as can other forms of protein powder. Of course, less water can be added above the vital mol of water per mol of ribose to ensure sweetness. The resulting sweet taste is well suited for candy.

Besides forming “rock candy”, even flavored, other candy containing ribose may omit the need for using ordinary table sugar, fructose, glucose, and other such foods that are harvested from plants. To substitute D-ribose monohydrate for them by either putting it directly into the composition in its viscous form, heated or not, or a concentrated aqueous solution of the monohydrate made by adding extra water, more or less depends on the texture desired. Either somewhat more or less of the water will still make a satisfactory mix when added to the other ingredients but will require discretion by the confectionary to make it correctly for the type of candy desired. Of course, the mixing can be facilitated by doing so at elevated temperatures such as 150° F. D-ribose powder is expensive, so the target of such products preferably would be to those who are taking ribose at the present time or who could be induced to try ribose for its health reasons.

These would be those who have heart trouble and those who do strenuous exercise. For both of these groups, having a protein as part of the candy or confection would be a benefit and whey protein would be a plus. Of course, soy powder could also be used, but whey protein would be more positive to muscle builders and would not be much more expensive than soy when compared to the cost of ribose. Then covering means, such as using a chocolate coat to finish the bar would be next. Finally wrapping means can be done as is done conventionally at candy confectioneries. Even though we are disclosing the most healthful possible candy bar, it may be necessary to add ingredients that are not considered to be as healthy in order to control costs and make the consistency of the candy mix acceptable for the type of candy it simulates. Therefore, it may be that D-ribose monohydrate is to be added to a conventional candy mix as merely an addition to it. Its sweetness and ability to mix into candy ingredients, the same way that ordinary sugar can, make it very compatible, but ribose “rock candy” is simplest and purest.

Now the endothermic chemical reactive nature of anhydrous ribose, certainly apparent in vivo, has become just as apparent in vitro as disclosed in patent application Ser. No. 10/886,070, with two new molecules disclosed there. At elevated temperatures in this disclosure even more new molecules have become apparent. The question then should arise, what kind of molecules should be targeted to react with anhydrous ribose in vitro at elevated temperatures? Obviously, purines, pyrimidines, pyrroles, and amino acids should be considered as likely, as well as non-nitrogenous carboxylic acids such as those of the tri-carboxylic acid cycle, including pyruvic acid and their salts, even sorbic and maleic acids. Other fatty acids would be candidates. The fact that the endothermic reaction can occur at room temperature with a nitrogenous ring structure, a pyrrole ring being part of the molecule that is one of the reactants in the sodium PCA reaction, it also reacts at elevated temperatures with straight chain amino acids such as arginine and carnitine. This means that it not only reacts at room temperature with some liquids, as disclosed in patent application Ser. No. 10/886,070, but at elevated temperatures with many solids as is being disclosed here, and we would assume that these are endothermic reactions also.

Anhydrous D-ribose can irreversibly alter the composition of many nutrients or food substance it comes into contact with, especially with mixing either at room temperature or at elevated temperatures such as 150° F. Monohydrate D-ribose which anhydrous D-ribose becomes is also a precursor of new molecules such as the reaction with L-arginine. If some of these new molecules prove valuable, the monohydrate may be a better precursor of them by not waiting for the anhydrous D-ribose to form the monohydrate as it appears to do. In the case of arginine, the monohydrate makes a more uniform product, but the new arginine-ribose compound formed by each appears to be the same with the same aqueous solubility. The same fact appears to be true with carnitine and even with niacin.

With respect to ribose compounds, the term is loosely used by those skilled in the art. Ribose is a carbon-containing molecule and such a molecule is made up of more than one kind of atom. On the other hand, the definition of a compound really depends on whether it has a chemical or pharmaceutical basis. If the in vitro endothermic nature of ribose is ignored, it could fit either one, being both a chemical and a pharmaceutical compound or a mix of chemicals. This paradox can readily be explained by citing Ribova et al. in U.S. Pat. No. 6,071,888, “Composition for Treating Cancer”, where ribose is claimed for such use in a pharmaceutical mix of several molecules or as the substances of them, which is what a composition is. Its potential conflict with respect to this present disclosure is that the simple term “ribose” is disclosed as one of the ribose compounds. If ribose indeed is one of the compounds, is it the same as D-ribose recovered by fermentation with corn syrup? The patent doesn't say so. If it is, it would no longer be the ribose of that patent but the one of this disclosure since when it is mixed with water it becomes ribose monohydrate. Water is in Ribova's compositions. Furthermore, as disclosed herein, because of the endothermic chemical reactions that anhydrous D-ribose undergoes in vitro, new ribose chemical compounds, ribosides or ribosyls if not something else, have been disclosed herein as a result of this inventor's discovery. These are new in vitro molecules, not old ones that are part of the in vivo chemistry in the body and its natural nutrients, as cited by Ribova. Since fermentation-derived endothermic D-ribose, in vitro with respect to mammals, is used herein but not with Ribova, their patent fails to disclose basic chemistry.

This disclosure is about the in vitro chemistry of anhydrous D-ribose, by which endothermic chemical reactions result in many new molecules, the proper labeling to consumers, but not the chemistry of its manufacture which was done previously to make the free molecule available for this disclosure. The chemistry of this disclosure starts when the anhydrous free molecule is designated to be consumed by or applied to living beings. Wholesale shipments of anhydrous D-ribose by its manufacturers to nutrient packagers and their distributors may require FDA oversight as to purity and safety, but is not governed by this disclosure. On the other hand, labeling for consumers requires the basic information presented in this disclosure, including understanding what the anhydrous powder becomes with heat and cooking so that truth in labeling follows federal and state regulations.

The question is posed as just who is skilled in the art with respect to the use of the hydrate of D-ribose? It must be kept in mind that ribose monohydrate is sort of a “schizophrenic” molecule in that it has three forms. First there is the opaque soft paste-like solid formed by adding a mol of water to a mol of the anhydrous powder at room temperature resulting in an obvious endothermic chemical reaction as disclosed above. If this substance is then heated it becomes an amber translucent fluid and when allowed to cool back to room temperature it retains its viscous translucence but over days it reforms opaque components. On the other hand, if the anhydrous powder is directly heated to the same temperature, it forms a similar amber translucent fluid but when this substance is cooled to room temperature it more quickly becomes a clear harder solid, then more slowly it becomes more opaque and crystalline-like. Obviously this molecule, changing to different physical forms at different speeds, was never physically described before, and is new art with respect to chemistry. The other problem is it is still new at the highest intellectual level for the preferred medical target for this molecule, those afflicted with heart failure. When it is realized that an article titled “Is the Failing Heart Energy Starved?” by Joanne S, Ingwail of Brigham and Women's Hospital, Harvard Medical School and Robert G. Weiss, John Hopkins Hospital and University School of Medicine, Cardiology Division, in the publication “Circulation Research”, July 2004, copyrighted by the American Medical Association, Inc., despite such a pedigree, the authors ask, “Is it possible to intervene and remodel the pathways for ATP synthesis? . . . because glucose utilization is more efficient at generating ATP per O₂ consumed, it is thought that such a substrate switch is likely adaptive and thus beneficial.” The fact is that these authors, obviously skilled in the therapeutic art, did not even realize ribose was being marketed to shorten the pathway. Thus obvious skill in the art is in very short supply both in biochemistry and cardiac medicine with respect to the in vitro anhydrous powder, let alone the hydrate.

While we have mostly discussed this disclosure as it applies to human beings, the same endothermic chemical reactions of in vitro anhydrous D-Ribose forming various hydrates including monohydrates apply to other animals and products designed for them.

Finally, while we have used the common term, ribose, in this disclosure, it is not limited to ribose, and includes any 5-carbon precursor or substitute of ribose, including D-ribose, ribulose, xylitol and xyulose if they can be a reactant in an endothermic chemical reaction with water and form new molecules either at room temperature or with heat.

While particular variations of the present invention have been described, it will be obvious to those skilled in the art that changes and modifications may be made without departing from my invention in its broader aspects of producing endothermic chemical reactions with the free molecule of anhydrous D-ribose and other molecules in vitro both at room and elevated temperatures.

What is claimed:

1. An endothermic chemical reaction employing a five-carbon atom pentose, usually referred to as ribose but any similar reactive molecule, that will engage in such a reaction forming new molecules.

2. The reaction according to claim 1 in which anhydrous D-ribose is the form of the ribose used.

3. The reaction according to claim 1 in which the monohydrate of D-ribose is formed at room temperature with water.

4. The reaction according to claim 3 in which the monohydrate of D-ribose can be formed and packaged by itself.

5. The reaction according to claim 3 in which the monohydrate of D-ribose formed first is mixed with other nutrients or food.

6. The reaction according to claim 1 in anhydrous D-ribose is mixed completely with another nutrient or food powder followed by one mol of water per mol of ribose added and the contents again thoroughly mixed.

7. The reaction according to claim 6 in which more than one powder is mixed with anhydrous D-ribose

8. The reaction according to claim 1 in which the resultant molecule is mixed with reactive molecules at elevated temperatures.

9. The reaction according to claim 8 in which the resulting molecules are either ribosides or ribosyls.

10. The reaction according to claim 8 with one or more molecules that result in the formation of new molecules not ribosides or ribosyls.

11. The labeling of anhydrous D-ribose for sale to consumers warning that it is unstable when heated with other substances, including with cooking.

12. The subjection of anhydrous D-ribose in its powder form by itself to elevated temperatures.

13. The invention according to claim 12 in which the molecule formed is ribose monohydrate.

14. The invention according to claim 12 in which a compact amber hard solid is formed.

15. The invention according to claim 14 in which said amber hard solid is ribose monohydrate.

16. The invention according to claim 12 in which one of the temperatures is 150° F.

17. The invention according to claim 12 in which the required source water is water vapor

18. The invention according to claim 17 in which water vapor is in the form of steam.

19. The invention according to claim 12 made into forms appearing like rock candy with or without flavoring.
